(19) United States
(12) Patent Application Publication
(10) Pub. No.: US 2023/0293018 A1
(43) Pub. Date: Sep. 21, 2023

(54) WHOLE BLOOD SAMPLING AND MONITORING DEVICE, METHOD AND SOFTWARE

(71) Applicant: Liron BEN-BASHAT BERGMAN, Binyamina (IL)

(72) Inventors: Liron BEN-BASHAT BERGMAN, Binyamina (IL); Ron BELOSSESKY, Ra'anana (IL); David SALTON, Hod Hasharon (IL); Gilad COHEN, Aviel (IL)

(21) Appl. No.: 18/010,621

(22) PCT Filed: Jun. 14, 2021
(86) PCT No.: PCT/IL2021/050716

§ 371 Date: Dec. 15, 2022

Related U.S. Application Data
(60) Provisional application No. 63/039,758, filed on Jun. 16, 2020.

(51) Int. Cl. A61B 5/02; A61B 5/00; A61B 5/157; A61B 5/15; A61B 5/1455; A61B 5/145; A61B 5/155

(52) U.S. Cl. CPC A61B 5/02042; A61B 5/746; A61B 5/157; A61B 5/150992; A61B 5/150099; A61B 5/14557; A61B 5/14546; A61B 5/14539; A61B 5/155; A61B 5/7275; A61B 5/15003

(57) ABSTRACT

The present invention provides systems and methods for monitoring whole blood drawn from a mammalian subject, the system including a fluid delivery device in fluid connection, at a first end, with a vein (or other blood vessel) of the subject, a valve device at a second end of the fluid delivery device, a whole blood monitoring apparatus for measuring a hemoglobin count for example in a blood sample conveyed by the fluid delivery device from the mammalian subject and a processor adapted to analyze data received from the hemoglobin monitoring apparatus to monitor the subject and further to detect if the subject is suffering from internal bleeding.

### FIELD OF THE INVENTION

The present invention relates generally to blood sampling devices and methods, and more specifically to methods and apparatus for blood analysis and blood monitoring.

### BACKGROUND OF THE INVENTION

Internal bleeding is a large cause of death. Failure to detect blood loss in real-time is a leading cause for medically preventable death. Fast diagnosis and control of bleeding is critical for the prevention of irreversible cell changes and severe damage to vital organs during hypovolemic shock. Evaluation of the actual blood loss and diagnosis of the hemodynamic status is a great challenge for medical staff. This is even a greater challenge in accidents, sports injuries or in military combat.

More than 25% of the injuries during battle include hemorrhage, while 10% of them include internal bleeding requiring immediate evacuation. It is extremely hard to diagnose blood loss before 30% of blood volume is lost, leading to changes in pulse, blood pressure and consciousness.

The monitoring of hemoglobin (Hgb) levels and pulse can provide an alert regarding blood loss and identify cases of undiagnosed or underestimated internal bleeding. Recent studies show that following massive bleeding changes in Hgb levels can be noticed in the measured blood within less than half an hour of the incident, due to rapid fluid shift to the inner vascular space. This contrasts with trauma and surgeon paradigm.

Many trauma patients suffer from internal bleeding, due to falls, vehicle accidents, gunshot wounds and other types of traumas. Many trauma patients die or become unconscious due to internal bleeding. Internal bleeding is one of the most serious consequences of trauma. Usually, the bleeding results from obvious injuries that require rapid medical attention. Internal bleeding may also occur after a less severe trauma or be delayed by hours or days.

Internal bleeding may also occur in other cases such as surgeries or complicated labors.

In many cases, the internal bleeding results from non-obvious internal injuries. These can be lethal, if not detected and treated.

To date, there are very few reliable methods, if any, for real-time diagnosis of internal bleeding in a patient. Thus, all too often, by the time the patient is diagnosed as suffering from internal bleeding, significant damage can be induced to the brain or other organs, or the patient may be dead.

There, thus remains an urgent need to develop improved methods and apparatus for detecting internal bleeding in a human subject.

There remains an urgent need for improved methods and apparatus for detecting and quantifying blood loss in mammalian subjects.

### SUMMARY OF THE INVENTION

It is an object of some aspects of the present invention is to provide improved methods, devices and systems for a monitoring blood level over extended periods of time in a mammalian subject.

It is another object of some aspects of the present invention is to provide improved methods and systems for detecting internal bleeding in a mammalian subject.

In some embodiments of the present invention, improved methods and apparatus are provided for drawing blood over many hours from a human subject.

In some embodiments of the present invention, improved methods and apparatus and devices are provided for optical analysis of whole blood from a mammalian subject.

In some further embodiments of the present invention, improved methods and apparatus and devices are provided for continuous or semi-continuous optical analysis of whole blood from a mammalian subject over many hours.

In some embodiments of the present invention, improved methods and apparatus are provided for detecting real-time internal bleeding in a human subject.

In other embodiments of the present invention, a method and system is described for providing continuous or semi-continuous monitoring of a human subject to detect internal bleeding or other whole blood indices. The invention further comprises systems, methods and devices for controlled monitoring, personalized monitoring and remote monitoring of blood parameters.

In additional embodiments of the present invention, a system is provided for detecting blood loss in a mammalian subject, the system including some or all of:
a) a fluid delivery device in fluid connection, at a first end, with a vein (or other blood vessel) of the subject—catheter,
b) a valve device at a second end of the fluid delivery device—connected to the monitoring apparatus and to an infusion bag (saline only or saline with heparin);
c) a hemoglobin monitoring apparatus for measuring a hemoglobin count in a blood sample conveyed by the fluid delivery device from the mammalian subject; a processor adapted to analyze data received from the hemoglobin monitoring apparatus to detect if the subject is suffering from at least one of blood loss and internal bleeding;
d) a blood parameter monitoring apparatus for monitoring real-time values of blood parameters, such as lactic acid, glucose, pH, viscosity, dissolved oxygen, carbon dioxide, platelet count and many other optional blood parameters.
e) a pump drawing the blood from the fluid delivery device through the monitoring apparatus to waste;
f) a hemoglobin monitoring device includes: 1. a thin plastic flow through cuvette with parallel flat surfaces 2 a light source 3. An optical sensor including a photodiode;
g) a wearable apparatus allowing fixing the device stably to the patient's body; and
h) software to enable algorithms as described herein.

In further embodiments of the present invention, the processor is further adapted to provide an alarm if the subject is suffering from at least one of blood loss and internal bleeding or any other blood indicator alarm it was programmed to test and alert for.

In further embodiments of the present invention, the invention provides systems and methods for early detection of body malfunctions in a patient based on real time monitoring of blood parameters from a catheterized patent, indicative of changes of state in the human body.

More particularly, the present invention relates to a diagnostic method, system and apparatus for rapidly detecting, at least one change in a trend of a blood parameter indicative of a body malfunction.

Yet more particularly, the present invention relates to a diagnostic method, system and apparatus for real-time detection of at least one change in a trend of a blood parameter indicative of a body malfunction.

Additionally, the present invention relates to a diagnostic method, system and apparatus for real-time detection of internal bleeding, detected by at least one change in a trend of a blood parameter.

According to some embodiments of the present invention, the blood parameter includes a hemoglobin level.—in other cases may be glucose, natrium, sodium bicarbonate, creatinine, oxygen saturation, blood pH. lactate etc.

Additionally, the present invention provides a system for the automatic and continuous monitoring of hemoglobin and pulse from a patient—absolute values and changes.

### EMBODIMENTS

1. A system for monitoring whole blood in a mammalian subject, the system comprising:
   a) a fluid delivery device in fluid connection, at a first end, with a vein of the subject;
   b) a valve device at a second end of said fluid delivery device;
   c) a hemoglobin monitoring apparatus for measuring a hemoglobin count in a whole blood sample conveyed by said fluid delivery device from said mammalian subject; and
   d) a processor adapted to analyze data received from said hemoglobin monitoring apparatus to detect changes in said hemoglobin count of said subject over time.
2. A system according to embodiment 1, wherein the processor is further adapted to provide an alarm if the subject is suffering from at least one of internal bleeding and blood loss.
3. An optical absorbance analysis device analyzing whole blood wherein the device is attached to a catheter and uses small quantities of blood such as hundreds of microliters and less, wherein the device is operative to analyze blood indicators such as hemoglobin.
4. A system for withdrawing blood from the body through a catheter, wherein the system draws blood by command sent from an automatic artificial intelligent system and wherein the system is disposable or partially disposable and works for at least 6 or 12 hours.
5. A system according to embodiment 4, wherein the system draws blood via a pump and wherein the pump works in pulses with a time control via a blood sensor and an artificial intelligence system.
6. A system according to embodiment 5, further comprising an optical sensor including at least one LED (Light Emitting Diode).
7. A system according to embodiment 6, wherein hemoglobin is detected at 550 nm, and wherein at least one LED outputs radiation at around 550 nm.
8. A system according to embodiment 7, wherein the sensor is a photodiode placed in front of the LED and therebetween is disposed a cuvette containing the blood sample. The sensor has an amplifier. The sensitivity is determined when the cuvette is clean. Working point is in the middle of the dynamic range of the sensor. Light intensity changes until it reaches the initial determined working point.
9. A diagnostic method for detecting at least one change in a trend of a blood parameter indicative of a body malfunction, the method comprising continuously or semi-continuously monitoring at least one blood parameter selected from at least one of: a hemoglobin level, a lactate level, a glucose level, an albumin level, an oxygen level, a sodium level, a potassium level, and pH and combinations thereof of a catheterized patient; whereby at least one dynamic trend is monitored so as to detect one or more changes in said at least one dynamic trend to indicate said body malfunction in said patient.
10. A diagnostic method according to embodiment 9, comprising semi-continuously monitoring a hemoglobin level of said catheterized patient.
11. A diagnostic method according to embodiment 10, comprising continuously monitoring, a hemoglobin level said catheterized patient.
12. A diagnostic method according to embodiment 11 wherein said monitoring is carried out less than once every hour.
13. A diagnostic method according to embodiment 12, wherein said continuous monitoring is carried out less than once every half hour.
14. A diagnostic method according to embodiment 13, wherein said continuous monitoring is carried out less than once every ten minutes.
15. A diagnostic method for detecting at least one change in a trend of a blood parameter indicative of a body malfunction, the method comprising:
   a. monitoring and transmitting at least one blood parameter of a catheterized patient; and
   b. detecting at least one of a hemoglobin level, a sodium level, an oxygen level, a lactate level, a potassium level, a pH and combinations thereof in the blood of said catheterized patient;
   whereby at least one dynamic trend is monitored so as to detect one or more changes in said at least one dynamic trend to reflect at least one of internal bleeding, external bleeding and combinations thereof in said patient or other body malfunctions projected.
16. A diagnostic method according to embodiment 9, further comprising providing an alarm means if internal bleeding is detected.
17. A device for real-time monitoring of whole blood in a mammalian subject, the device comprising:
   a) a valved element for receiving a whole blood sample from said human subject;
   b) a hemoglobin monitoring apparatus for direct real-time measurement of a hemoglobin count in said whole blood sample conveyed by said valved element from said mammalian subject; and
   c) a processor adapted to analyze data received from said hemoglobin monitoring apparatus to detect changes in said hemoglobin count of said subject over time.
18. A device according to embodiment 17, wherein said device is an optical absorbance analysis device, configured to analyze said whole blood sample and wherein said whole blood sample is of a volume of less than 150 microliters.
   The present invention will be more fully understood from the following detailed description of the preferred embodiments thereof, taken together with the drawings.

### BRIEF DESCRIPTION OF THE DRAWINGS

The invention will now be described in connection with certain preferred embodiments with reference to the following illustrative figures so that it may be more fully understood.

With specific reference now to the figures in detail, it is stressed that the particulars shown are by way of example and for purposes of illustrative discussion of the preferred embodiments of the present invention only and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1A is a simplified schematic illustration of a system for diagnosing blood indices by a single point test, in accordance with an embodiment of the present invention;

FIG. 1B is a simplified schematic illustration of a system for real-time monitoring of whole blood, in accordance with an embodiment of the present invention;

FIG. 2 is a simplified pictorial illustration showing a device for monitoring whole blood, in accordance with an embodiment of the present invention;

FIG. 3A is a simplified pictorial illustration showing a device for monitoring whole blood for mounting on an arm, in accordance with an embodiment of the present invention;

FIG. 3B is a simplified pictorial illustration of a perspective three-dimensional view of the device of FIG. 3A, in accordance with an embodiment of the present invention;

FIG. 3C is a simplified pictorial illustration showing a device for mounting and positioning on an arm, in accordance with an embodiment of the present invention;

FIG. 4A is a top view of another example of the device for monitoring whole blood, in accordance with an embodiment of the present invention;

FIG. 4B is a side view of the device of FIG. 4A for monitoring whole blood, in accordance with an embodiment of the present invention;

FIG. 5 is a simplified pictorial illustration showing another device for monitoring blood of a patient as a single point test, in accordance with an embodiment of the present invention;

FIG. 6A is a simplified pictorial illustration showing an open cuvette for receiving whole blood, in accordance with an embodiment of the present invention;

FIG. 6B is a simplified pictorial illustration showing a closed cuvette for receiving whole blood, in accordance with an embodiment of the present invention;

FIG. 7 is a simplified schematic illustration showing a device for monitoring blood of a patient, in accordance with an embodiment of the present invention;

FIG. 8 is a simplified schematic illustration of an electrical system in the device of FIG. 7, in accordance with an embodiment of the present invention;

FIG. 9 is a simplified flow chart of a method for decision making in monitoring blood flow in a patient, in accordance with an embodiment of the present invention;

FIG. 10 is a simplified sensor flow chart of a method for real-time monitoring of whole blood flow in a patient, in accordance with an embodiment of the present invention;

FIG. 11 is a calibration graph of hemoglobin concentration against voltage of a rabbit, in accordance with an embodiment of the present invention;

FIG. 12 is a graph presenting experimental results of monitoring voltage over time after device rinsing to show that optical clarity is consistent during the entire usage period of the device, in accordance with an embodiment of the present invention, and

FIG. 13 is a graph showing experimental results of monitoring real-time hemoglobin concentration in a patient with infusion over time to study hemodilution behavior, in accordance with an embodiment of the present invention; and

FIG. 14 is a simplified graph of hemoglobin concentration versus voltage of whole blood in mammals, in accordance with an embodiment of the present invention.

In all the figures similar reference numerals identify similar parts.

### DETAILED DESCRIPTION OF THE EMBODIMENTS

In the detailed description, numerous specific details are set forth in order to provide a thorough understanding of the invention. However, it will be understood by those skilled in the art that these are specific embodiments and that the present invention may be practiced also in different ways that embody the characterizing features of the invention as described and claimed herein.

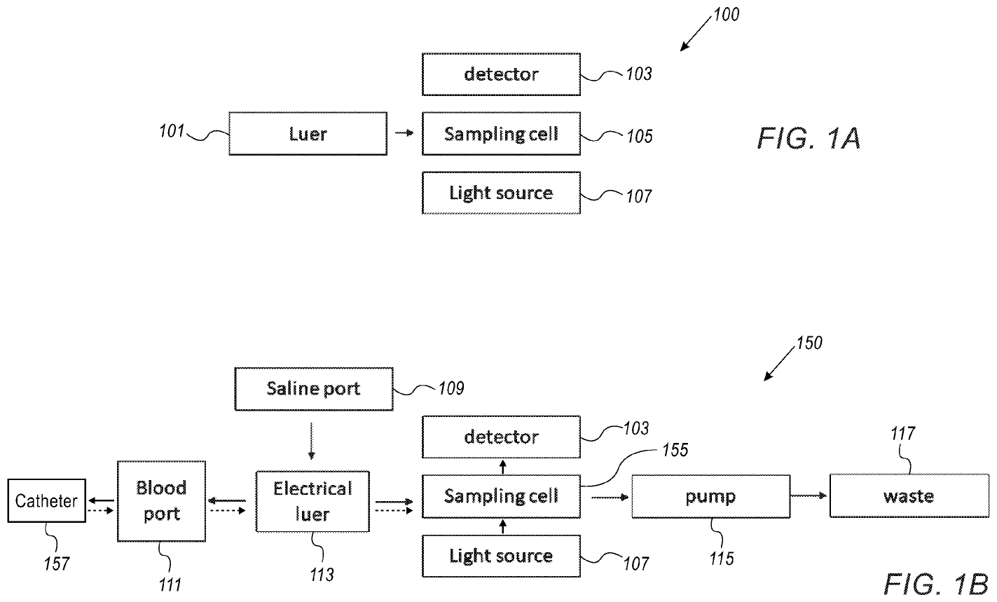

FIG. 1A is a simplified schematic illustration of a system 100 for diagnosing blood indices by a single point test, in accordance with an embodiment of the present invention.

FIG. 1A shows one configuration, which is possible for the device and presents the device working concept. A single point device 100 is configured to allow to test whole blood sampled externally and inserted into a suitable luer, valve or stopcock 101 (these terms may be used interchangeably). The Luer receives whole blood or another type of liquid sample, previously removed from a human or animal/mammalian subject. The whole blood from the luer is inserted by pressure or by capillary forces into a sampling cell 105 where it is radiated using a light source 107. Light of a specific wavelength is then detected by a photodiode detector or other detector 103 and analyzed for blood indices values. The term “branula”, catheter and cannular are used to mean a narrow tube for insertion into a bodily cavity, such as a vein.

FIG. 1B is a simplified schematic illustration of a system/device 150 for real-time monitoring of whole blood, in accordance with an embodiment of the present invention. FIG. 1B shows a real-time monitoring version of the device. This configuration is mounted on a patient and involves a pump 115 which draws blood from a catheter 157 connected to a blood port 111 into a sampling cell 155 or infusion liquid through a saline port 109 for rinsing the device, using the electrical luer 113. The electrical luer 113 is connected to the catheter through the blood port 111 and to an infusion bag (not shown) through a saline port 109. This allows automatic switching between three states: infusion dripping to the body (continuous arrows) whenever a blood test is not being performed, blood pumping through the device (broken arrows) and infusion pumping to the device for rinsing (continuous arrows). The blood and saline drawn by the pump are transferred to the waste receptacle 117.

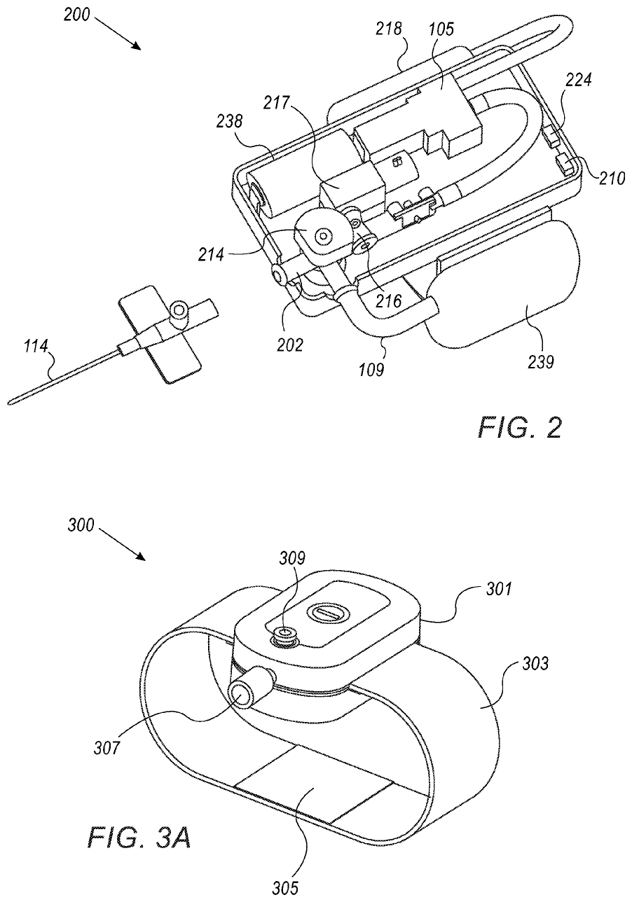

Reference is now made to FIG. 2, which is a simplified pictorial illustration showing a device 200 for monitoring blood of a patient, in accordance with an embodiment of the present invention.

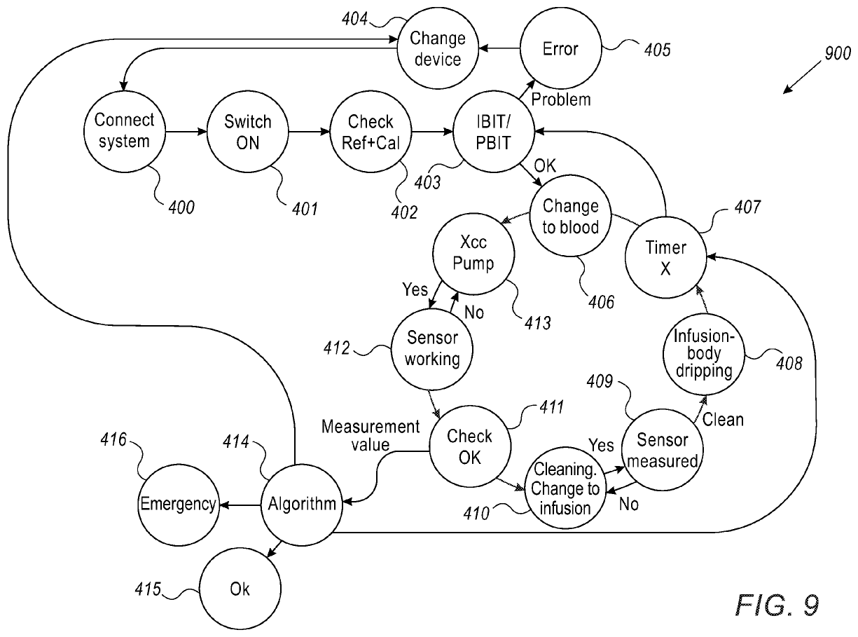

According to some embodiments, the present invention includes an analytical disposable miniature device, assembled on a Venflon (or intravenous cannula) communicating with a remote computerized station. The device automatically and frequently draws a minimal amount of blood (few hundreds microliters) for measuring hemoglobin (Hgb) levels. Hemo-dilution is considered for Hgb correction, as may be needed, using an algorithm (as described herein—see FIG. 9 for example) and more frequent testing of the patient's blood.

Additionally, pulse is measured as a complementary index using a pulse meter (not shown) attached to the device. The device is configured to detect and/or diagnose dangerous health situations in a patient/subject. This is performed by integrating between the pulse and Hgb changes, monitored over time. According to additional embodiments, changes in a patient's bodily function can be detected/diagnosed using Hgb monitoring alone. The device may be used as a platform for additional sensors for blood monitoring, such as oxygen, lactate, glucose, creatinine, blood pH and electrolytes blood level. The device may also be a part of fluid balance follow up.

The device is constructed and configured to be easily portable, inexpensive and consumes low power, due to its small size and its utility on basic modern technologies. The device is further constructed and configured to be durable in field conditions and simple to operate for easy accessibility. The measured Hgb and pulse data is sent at an adjusted frequency by Bluetooth (or other transmission method) to a main medical station (not shown) allowing the indices to be monitored and analyzed locally and/or remotely.

The present invention system and devices are constructed and configured to optimize real-time evaluation of a patient's condition. This allows for urgent evaluation followed by improved medical treatment.

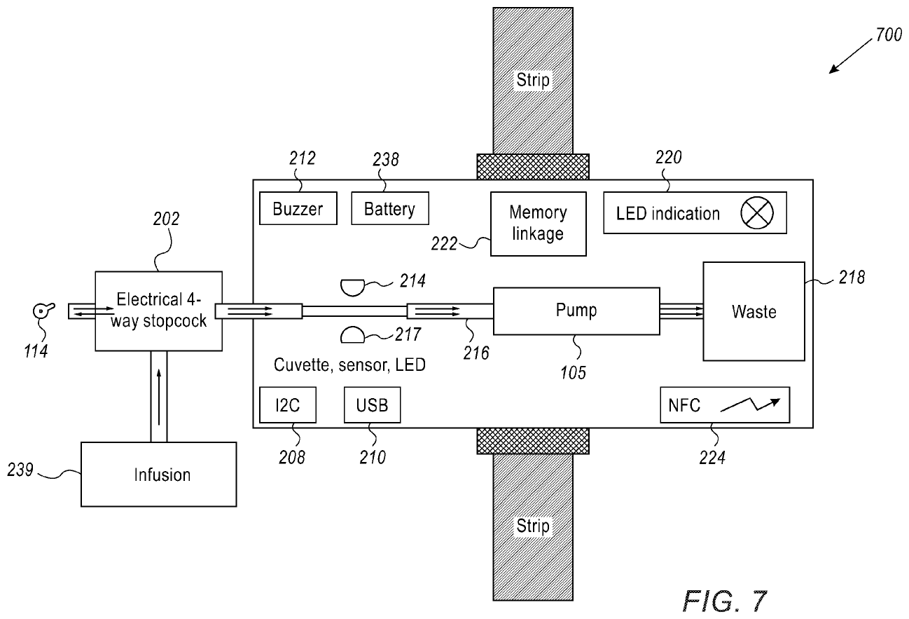

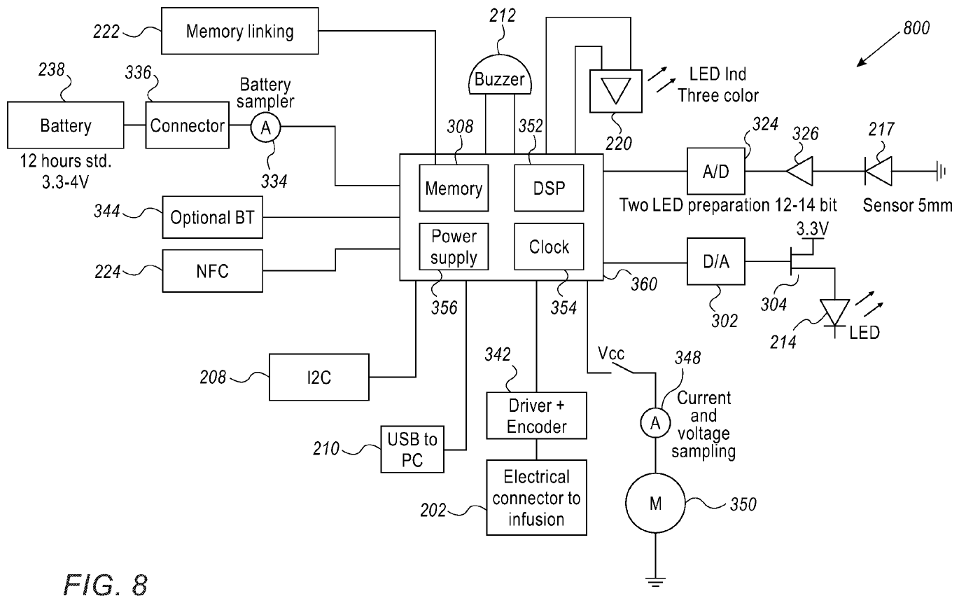

According to one embodiment as shown in FIG. 2, device 200 may be disposable. In one example, the following legend applies:— a DC pump 105, an infusion tube 109, configured to transfer an infusion liquid (not shown), for example from an infusion reservoir 239. A needle 114 is used to extract a whole blood sample from a patient/subject. A battery 238 provides energy to the device components including (as shown schematically in FIGS. 7 and 8) an integrated circuit 208, a USB port 210, an LED indicator 220 (also can be a display for displaying data). Data may also be transferred from the device via a near field communication element 224 to a computer, for example. The sample from the subject/patient is transferred from a needle via a connector 202. The connector may be, according to some embodiments, an electrical stopcock (FIG. 7). This enables the sample to optionally be treated with an infusion liquid (not shown from the infusion reservoir). A valve engine (also termed herein stopcock motor) 217 and tube 216 enables communication between the stopcock 214 and a pump 105, as well as from the pump to a waste reservoir 218. If a dangerous health situation in the patient/subject is detected and/or there is a device malfunction, a buzzer or alarm 212 is activated. Data may be stored in a local memory

The device in FIG. 2 uses LED for transmitting radiation, known to be absorbed by hemoglobin (Hgb). The radiation is detected by a photodiode detector. Whole blood (for testing) is drawn from the blood flow into the cuvette and then to a waste reservoir 218 by a needle (catheter) 114 using a DC pump 105. The data from the device is transmitted to the “display” via Bluetooth (BT) transmission 224 or other transmission method. The Bluetooth communication is bi-directional so the stationary unit can trigger the sampling as required by a medical staff.

The device shown in the figures (FIGS. 2, 7 and 8, for example) is powered ON by a switch, embedded in the device and receives power from a battery of 5V or less 238. The device alarms whenever there is a device fault or suspicion of hemorrhage using a buzzer 212. Whenever there is a need to change the device, all data can be transferred to the new device through the USB port 210.

The device includes an electrical stopcock 214 which switches between three states: infusion constant dripping to the body through the infusion port 109 connected to an infusion reservoir 239, rinsing the device using the infusion liquid and pumping blood from the body into the device through 202.

The frequency at which the patient's blood is sampled, is controlled by an automated switch, configured to turn the device battery on and off. More information regarding the electrical system can be found in FIG. 8.

In one embodiment, algorithms include:
a) correlating between changes in Hgb levels (with/without pulse) to detect health danger situations in the patient and blood sampling frequency;
b) automatically calculating Hgb levels of the patient to detect hemo-dilution of the patient; and
c) raising an alarm and/or alerting upon patient health danger situations.

In one embodiment the device is a one-use and/or disposable measurement device which is “patched to the patient”, vis-a-vis the current methods of separated measurement devices of blood samples which located at the POC (“Point of Care” i.e. patient's bed or mobile carriage).

In one embodiment, the measurement device is configured to obtain measurements in a continuous manner. In another embodiment, measurements are performed automatically every several minutes by blood is being vacuumed/pulled/extracted from the subject/patient and screened within the closed device. This is performed vis-à-vis current methods of taking a blood sample from a patient/subject and transferring/moving the sample to a separated detached measurement device.

In one embodiment the device is connected to an artery and the blood is drawn into the device due to pressure differences between the device and blood vessel or the blood is controllably pumped by a pump.

In one embodiment, the system is built from a disposable and portable device and a stationary unit (any computer or phone with a designated program). The disposable unit will be assembled on a Venflon.

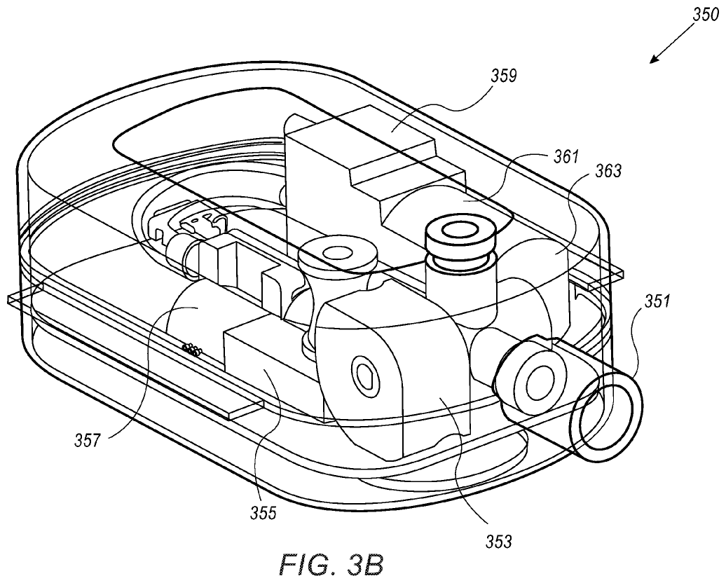

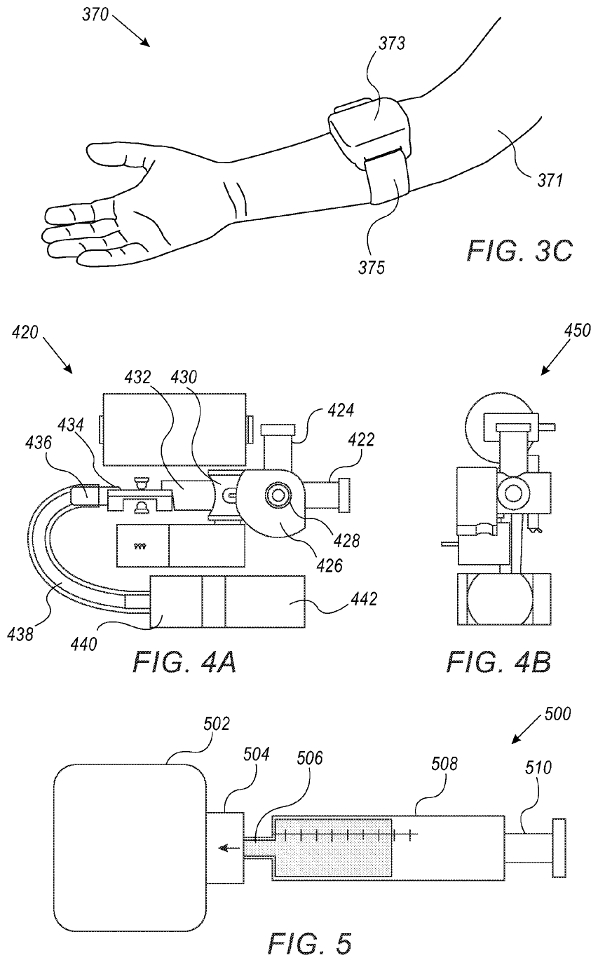

FIG. 3A is a simplified pictorial illustration showing a wearable device 300 for monitoring whole blood for mounting on a person's arm, in accordance with an embodiment of the present invention. This embodiment shows an external casing 301, a hand strap 303 with strap connectors 305. A connector 307 to a catheter (not shown) and a port 309 connected to a rinsing liquid (not shown). Legend—301 Device casing, 303 Hand strap, 305 strap connector, 307 Luer/port to connect to canula and 309 luer/port to connect to saline bag.

FIG. 3B is a simplified pictorial illustration of a perspective three-dimensional view 350 of the device of FIG. 3A, in accordance with an embodiment of the present invention. This figure shows a half transparent view of the device. The half transparent view in FIG. 3B shows the internal arrangement of the components in a device 350. A pump 361 and a pump casing 359 is shown in connection with an electrical valve 353 and its motor 357. A connector 351 to the catheter is seen. A waste bag/receptacle 218 may be flexibly inserted in the dead spaces of the device to receive waste (blood/saline) from the pump.

FIG. 3C is a simplified pictorial illustration showing a device 370 for mounting and positioning on an arm, in accordance with an embodiment of the present invention. Here, the figure shows how to fit a device 373 using suitable straps 375 in cases where it needs to be connected to the wrist 371 area of a human subject.

FIG. 4A is a top view of another device 420 for monitoring whole blood, in accordance with an embodiment of the present invention. Here, the components are arranged differently for the waste bag to be placed inside the device (instead of 218). And instead of 239 (infusion container), the device is connected to a standard external infusion bag, per the legend below.

FIG. 4B is a side view 450 of the device of FIG. 4A for monitoring whole blood, in accordance with an embodiment of the present invention. A luer 422 is configured to connect to a branula (114). A second luer 424 provides fluid communication to a saline bag (not shown). An electrical valve 426 with an electrical valve pivot axis 428 is activated by an electrical valve motor 430. A third luer 432 connects between a connector 436 and sampling cell 434. Saline may be provided for cleaning the device and/or dilution from the saline bag. The sample passes into a sampling cell 434. A pump 440 is used to mechanically convey the saline to the waste 442.

FIG. 5 is a simplified pictorial illustration showing another device 500 for monitoring blood of a patient, in accordance with an embodiment of the present invention. This may be a single test, single-use or disposable cuvette device. It includes only a disposable cuvette 502 with a sensor, a PCB and battery. The blood is inserted from a luer 504 into a cuvette 502 using a syringe 508 which connects to the luer 504 and pushes blood into the cuvette 502 via the syringe handle 510. A syringe 506 is used to obtain a blood sample from the patient. The sample may be of a volume of 0.15 mL or less, according to some embodiments.

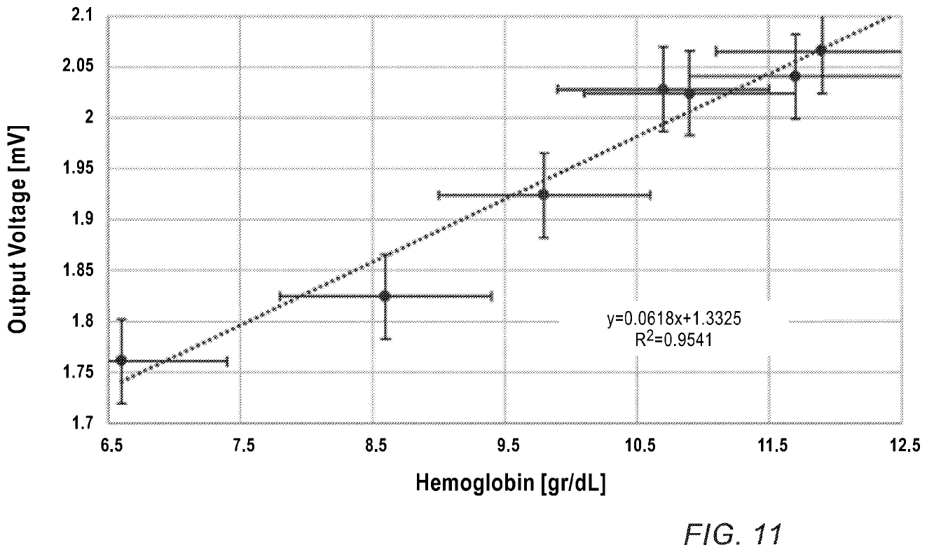

The blood sample is illuminated by the LED and the photodiode detects the intensity of light which passes the cuvette. The data is converted into Hgb concentration and is presented on the device screen. Then the cuvette is discarded. Some examples are shown in FIGS. 11-14 herein below.

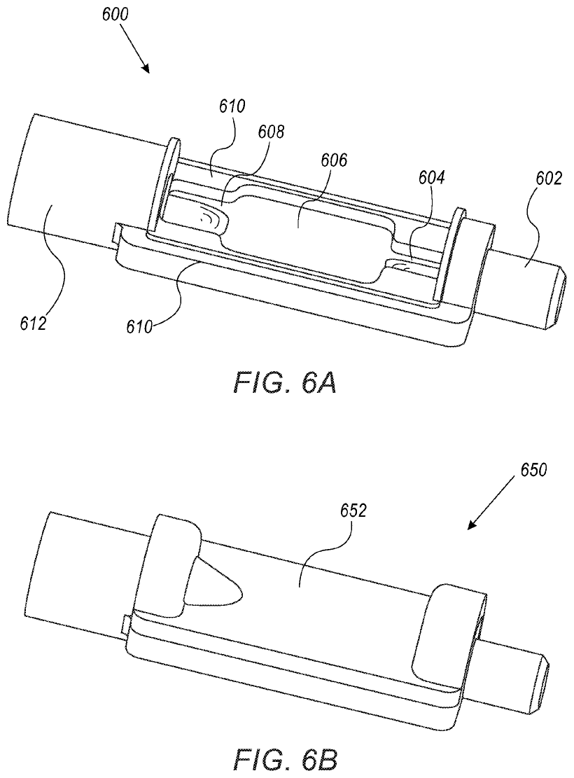

FIG. 6A is a simplified pictorial illustration showing an open cuvette 600 for receiving whole blood, in accordance with an embodiment of the present invention. FIG. 6B is a simplified pictorial illustration showing a closed cuvette 650 for receiving whole blood, in accordance with an embodiment of the present invention.

One non-limiting example of cuvette is shown in FIGS. 6A and 6B. The cuvette is comprised of two independently prepared parts which are later connected. A socket part 610 includes a half-circular cross-sectioned entry port/luer 602 (which receives whole blood from an external branula—not shown) to the cuvette in fluid connection with a narrow entry channel 604 to a rectangular cross sectioned sampling cell 606. Later, there is a narrow exit channel 608 receives fluid from the sampling cell to the waste. The channel changes gradually into a half circular cross sectioned exit channel 612. The cuvette further comprises a lid 652 (FIG. 6B).

FIG. 7 is a simplified schematic illustration showing a device 700 for monitoring blood of a patient, in accordance with an embodiment of the present invention. the device is constructed and configured to measure several independent blood parameters.

FIG. 8 is a simplified schematic illustration of an electrical system 800 in device 700 (FIG. 7), in accordance with an embodiment of the present invention. LED radiation 214 is detected by a photodiode detector 217. Whole blood for testing is drawn from the blood flow into a reservoir by a needle 114 using a dc pump 105. After the blood is tested it is passed to the waste container 218. The data from the device is transmitted to the “display” via Bluetooth transmission. The Bluetooth communication is bi-directional so the stationary unit can trigger the sampling as required by a medical staff.

A schematic diagram of the electrical elements, included in the portable unit of the device, is presented in FIG. 8. A sensor 217 and its electrical system components, an electrical stopcock 202 and a driver 342 are shown. Also shown is a PCB 360. The PCB may comprise many components, such as, but not limited to a power supply 356, a clock 354, a DSP 352 and a memory 308. An alarm system 220 and buzzer 212 are also seen. Also, the ability to use a memory link 222, an I2C 208, an Near Field Communication (NFC) 224, an optional BT 344 and a USB 210 to PC are presented. A battery 238 has a connector 336 and battery sampler 334, connected for inspection and control.

The setup of the electrical elements should not be deemed limiting. Many variations are possible. The device also may include a battery sampler 334, another optional BT 344, a current and voltage sampling element 348, one or more connectors 336, a pump motor 350, all in electrical connection with a power supply 356. A driver and encoder 342 may be used to control the infusion.

According to some embodiments, the present invention includes an analytical disposable miniature device assembled on a Venflon communicating with a remote computerized station. The device is constructed and configured to automatically and frequently draw a minimal amount of blood for measuring Hgb levels. Hemo-dilution will be considered for Hgb correction as needed. Additionally, pulse is measured as a complementary index using a pulse meter attached to the device. The device system will be able to diagnose health danger situations with high accuracy by measuring Hgb levels and changes monitored with time. The device is a platform for additional sensors for blood monitoring such as, but not limited to, oxygen, lactate, glucose and electrolytes blood level, natrium, sodium, bicarbonate, creatinine, oxygen saturation, blood PH, natrium, sodium, bicarbonate, creatinine, oxygen saturation, blood pH,

The device is aimed to be easily portable, cheap and will consume low power due to its small size and its utility on basic modern technologies. The device is planned to be durable at field conditions and easy to operate for easy accessibility. The measured Hgb data will be sent at an adjusted frequency by Bluetooth (or other communication method) to the main medical station allowing the indices to be monitored and analyzed. The methods of the present invention are directed to optimize evaluation of a patient's condition in real-time, thereby allowing urgency evaluation, followed by a better and more rapid medical treatment.

Reference is now made to FIG. 9, which is a simplified flowchart 900 of a method for decision making in monitoring blood flow in a patient, in accordance with an embodiment of the present invention.

The system is connected to the Venflon 400—with the strips/straps (optional) There may be another method for attaching the device to the patient. Initially, the device is switched ON in a switching on step 401. Then in a calibration step 402, a person using the device check reference values and calibrates the device. The device starts to work and automatically performs an Initializtion Built In Test (IBIT) process and calibration in a self-calibration step 403. The device draws a small amount of blood from the patient and in sampling steps 406, 413. Thereafter, in an activate algorithm step 412 and verifying step 411, the device is operative to start sampling through algorithm process.

If the sensor value is OK but outside the safe values of Hb levels for example, emergency procedure should take place (for example: buzz alert). The system continues to the cleaning stage to avoid blood coagulation in the sampling cell and total system in a cleaning device step 410.

After saline+heparin cleaning, the sensor checks if the sampling cell is clean by radiating it and checking if the signal reaches the voltage zero point in a sensor testing step 409. If cell is not clean, another cleaning procedure takes place in an infusion step 410. If thereafter, the cell is found to be clean, valve 202 (FIG. 8) switches to an infusion step 408 to drip to the patient's body. The whole measurement cycle is to be repeated when the timer is activated in a timer activation step 407. The timer is based on an artificial intelligent algorithm which decides the frequency of blood measurements as a function of Hb level and change in Hb level. Any time the system points at a functionality problem it goes back to a Periodic Built-In Test (PBIT) step 403 and provides an alert or an error may activate buzzer 212 (FIG. 8).

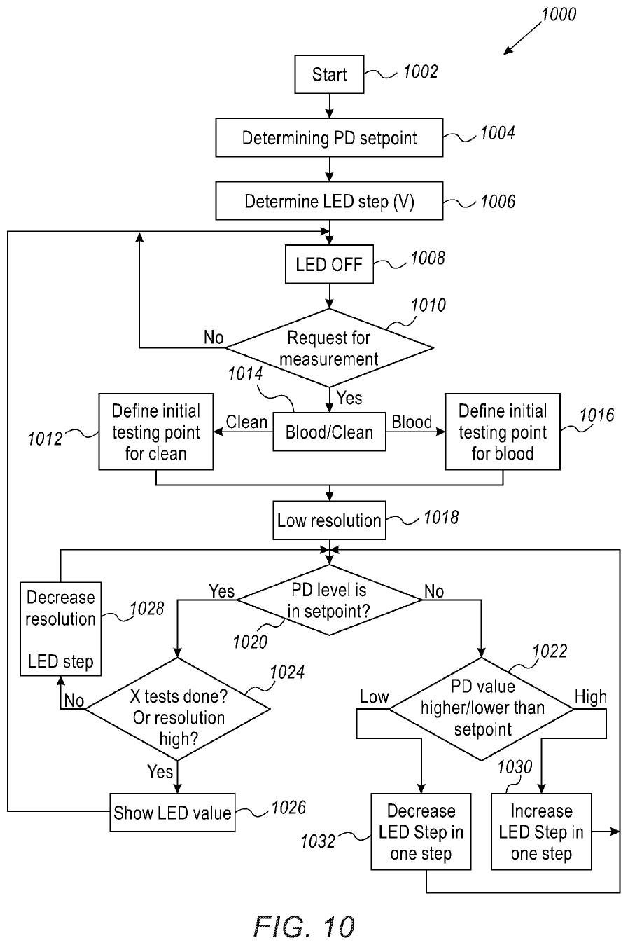

FIG. 10 is a simplified sensor flow chart of a method 1000 for real-time monitoring of whole blood flow in a patient, in accordance with an embodiment of the present invention;

The first step is initializing the sensor by start command in a start step 1002. Then the photodetector (PD) setpoint is being determined according to blood reception at the specific wavelength in a photodetector set point step 1004. This is followed by determining the right LED step according to the accuracy and time required, in a LED determining step 1006. Then the LED receives a command to turn ON the device in an LED activation step 1008. It then performs a measurement in a blood sample measuring step 1010. Then, it asks whether it is measuring blood or rinsing liquid in a checking liquid composition step 1014. There is a different initial testing point for each procedure (a defining initial testing point for cleaning step 1012/or define initial testing point for a blood procedure step 1016. After blood sampling or rinsing, the LED intensity of the radiation is changed in defined steps until the PD reaches its initially determined setpoint value. In the beginning, the LED steps change with a low resolution in a low resolution step 1018. If the PD level reaches its setpoint in a showing value step 1026, it means that X tests have been performed and sufficient resolution achieved. If not, decrease LED resolution 1028 and search again for PD setpoint 1018.

Thereafter, then the LED value is set in a setting step and sent to output steps (PD higher or lower than set point checking step 1022. If LED has not found a value which correlates to a PD setpoint value in an LED increasing step 1030 then its step size is lowered in one step (step 1032) or increased (step 1030) in accordance with the distance from a PD setpoint.

Turning to FIG. 11, there is seen a calibration graph of hemoglobin concentration against voltage, in accordance with an embodiment of the present invention. The system (technological sample) was tested on rabbits. A four hours experiment was conducted at the Veteran hospital at the Rambam Institute. The rabbit was put to sleep and was connected to a venflon through an artery line in the ear. A stopcock was connected to the venflon and allowed infusion dripping to the rabbit's body. The minimal amount of dripping was applied in order to keep the arterial line open through the experiment and in order to rinse the system after each blood drawing test.

FIG. 11 shows the device output voltage vs. rabbit hemoglobin levels (tested in hospital lab). There is a linear connection between the two. It is clear that the output voltage decreases with Hb levels. An initial behavior description is given by the trendline. These results imply that tracking after Hb levels can point on hemorrhage.

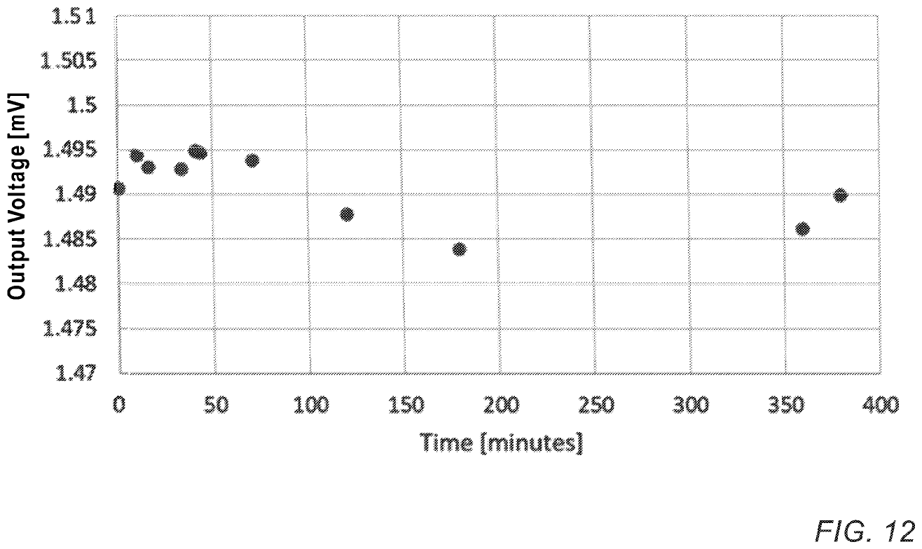

The ability of the system to remain optically clean after several blood draws along a four-hour experiment is presented in FIG. 12. The rabbit was bled by approximately 7 mL every several minutes. The system was rinsed using saline+heparin after every blood draw test. After the rinsing, the sensor tested the sample cell and checked if it is clean (return to zero-point voltage). It is seen that the sensor voltage value is repeatable (within a small error) through the entire experiment.

Reference is now made to FIG. 12, which is a graph showing experimental results (same experiment as FIG. 11) of monitoring voltage over time after device rinsing. The purpose of this test is to show that there is no blood residue in the system after rinsing which might affect the next blood test. The graph shows that the cuvette voltage readings remain constant within experimental error along the entire four-hour experiment (return to zero-point voltage).

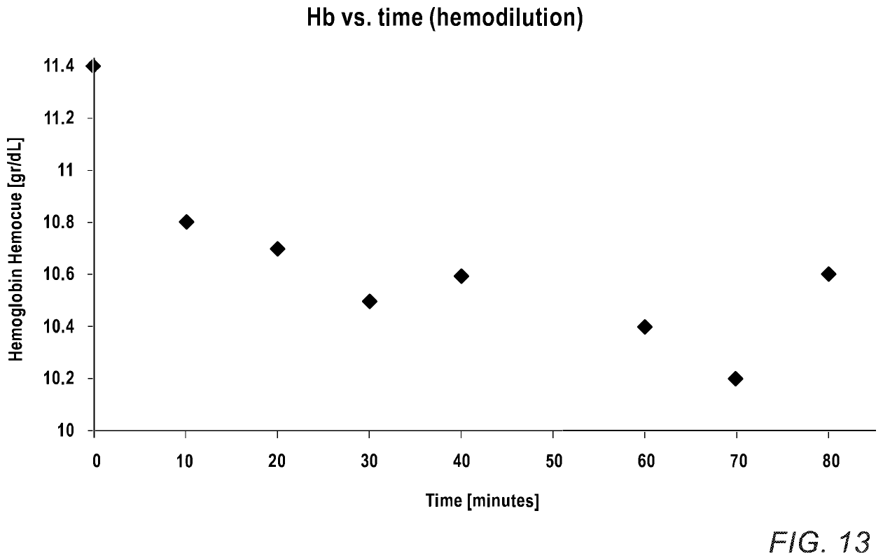

FIG. 13 is a graph showing the results of an experiment of Hb levels (tested by Hemocue device) during 1 liter of infusion dripping (saline) which lasted an hour to a patient. The system involves infusion dripping to the body, this can cause hemodilution which results is lower Hb levels. To allow a reliable testing of blood indices such as Hb levels, the hemodilution needs to be corrected. After 30 minutes the Hb levels reached a stable value. The correction of the Hb levels is approximately 1 gr/dL. Improved experiments may possibly improve the accuracy of the results. However, it can be generally concluded that hemodilution may be corrected by using the devices and methods of the present invention, including the application of a smart “real-time” algorithm, to provide results within a few minutes from the initial blood sample.

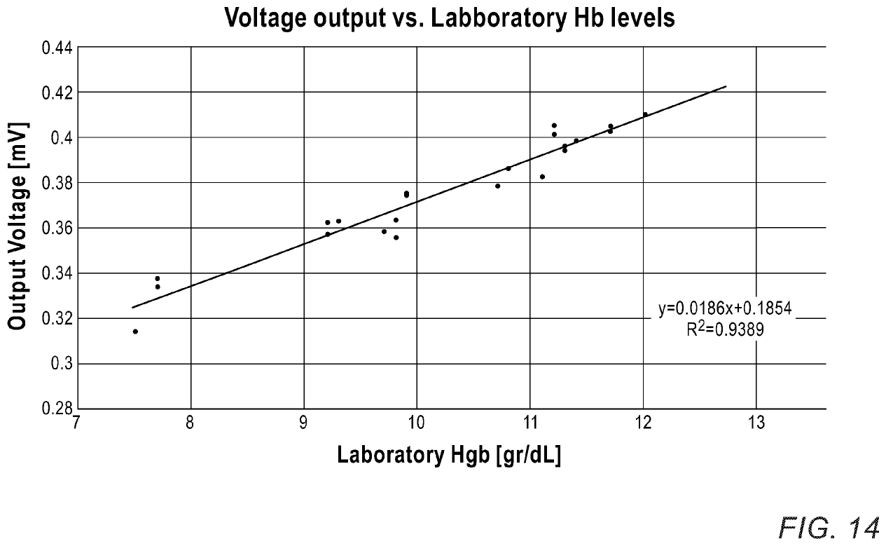

FIG. 14 is a graph of hemoglobin concentration versus output voltage of whole blood, in accordance with an embodiment of the present invention.

Fresh whole blood samples were drawn from female patients into test tubes and inserted into the cuvette by a syringe within 24 hours. Then the blood was illuminated by the LED and analyzed after the photodetector sensing. The test tubes were cooled to approximately 4° C. during the time gap between blood drawing and testing. Each blood sample was drawn twice so one tube was tested for Hb using standard laboratory test and the other by the current innovation. The setup of the current innovation was rinsed using saline and heparin after each test.

The parameters affecting the results include optical pathlength, cuvette optical transparency, light ray diameter and intensity, photodiode set-point and sensitivity, and stray light. Stray light is a function of air bubbles, red blood cells scattering, and cuvette boundaries. Light scattering is assumed to be the main reason for deviation from linearity at higher Hgb levels.

While the experiment conducted using a system connected to a rabbit's blood stream showed good linearity between detector output voltage and Hb levels, the results achieved for humans showed some discrepancy from linearity especially for Hb range of 10-13 gr/dL. The experimental setups of the later are more susceptible to light scattering and errors as it is remote from the body and less consistent. For example, there were air bubbles involved in these experiments and the cuvette was made of polished polycarbonate instead of quartz (less optically clear).

### Detailed Description of System Algorithms

1. Algorithm for Timing Blood Draw Intervals

Smart intelligent system:
Z -sensor error (3%), Y- 5 minutes, X - Hgb level
t=t0
withdrawing blood and measuring X levels
t1=t0+2Y (Y=2,3,4,...)
withdrawing blood and measuring X levels.
Calculating X(t1)−X(t0)
If
X(t1)−X(t0) > Z then withdraw blood in 2Y
If
X(t1)−X(t0) = Z then withdraw blood in 4Y
If
X(t1)−X(t0) < Z then withdraw blood in 9Y

Characteristics of the Devices of the Present Invention

They are wearable, cheap, disposable, accurate, automatic, remote monitoring and alerts devices. The devices of the present invention are constructed and configured to enable a diagnostic method for detecting at least one change in a trend of a blood parameter indicative of a body malfunction, the method comprising continuously or semi-continuously monitoring at least one blood parameter selected from at least one of: a hemoglobin level, an albumin level, an oxygen level, a sodium level, a potassium level, a lactate level and pH and combinations thereof of a catheterized patient; whereby at least one dynamic trend is monitored so as to detect one or more changes in said at least one dynamic trend to indicate said body malfunction in said patient.

Electrical Stopcock

In order to allow infusion transmission through the same Venflon as the device a unique stopcock is designed. The stopcock has three ports—one goes into the venflon, second to the device and third connected to the infusion.

Algorithms

2. Algorithm for Diagnosing Internal Bleeding and Alerting

```
  [X] - Hgb critical concentration, dX - Hgb critical change , M - critical
graph gradient
  t=0 : Measure and record Hgb levels - Start recording. If Hgb below X
gr/dL then alarm and continue.
  t=10 min: Measure and record Hgb levels - If Hgb below X gr/dL then
alarm and continue
  t=20 min: Measure and record Hgb levels - If Hgb below X gr/dL then
alarm and continue
  Calculate change in Hgb levels. - If Hgb change is larger than dX
gr/dL , alarm and continue
  If No change - continue.
    If Positive change - check again and continue
    If Negative change - generate graph Hb vs. time.
  t=Yx20 min (Y=2,3,4,...) : Measure and record Hgb levels - If Hgb
below X gr/dL then alarm and continue
  Calculate change in Hgb levels. - If change is larger than dX gr/dL then
alarm and continue
  If No change - continue.
    If Positive change - check again and continue. Check for error.
    If Negative change - continue graph. Calculate gradient. If gradient <
M alarm and continue. If not, continue.
```

3. Algorithm Process for Diagnosing Internal Bleeding Using Hgb Levels Analysis Under Hemodilution

```
  There are generally three cases to distinguish:
  No bleeding. Only infusion hemodilution
  Stable bleeding + infusion hemodilution
  Unstable bleeding - constant change in Hgb changing levels + Hemodilution
  Assumptions:
  Hemodilution becomes stable after 20 minutes.
  Maximal change in Hgb levels due to hemodilution is 1.2 gr/dL
  t=0 : Measure and record Hgb levels. If Hgb below X gr/dL then alarm and
continue.
  t=1 min : Insert infusion. Measure and record Hgb levels. If possible record
infusion rate.
  t= 10 min :_Measure and record Hgb levels - If Hgb below X gr/dL then alarm
and continue.
  t= 20 min :_Measure and record Hgb levels - If Hgb below _X gr/dL then
alarm and continue.
  Calculate graph parameters (Hgb vs. time) y=a1x+b or higher order: b= Hgb
(t=0), a= GRAD,
  t= 30 min :_Measure and record Hgb levels - If Hgb below X gr/dL then alarm
and continue.
  Calculate graph parameters y=a2x+b or higher order: b= Hgb (t=0), a=
gradients
  Compare results to previous check. If no change in gradients then continue to
check every ten minutes. If there is a change in gradient than calculate: a2 −a1 <= |1.2|
gr/dL then = Grad CORRECTION. Calculate graph with Grad CORRECTION and
correct future results. then continue. Else, Alarm.
  t= 40 min :_Measure and record Hgb levels - If Hgb below X gr/dL then alarm
and continue.
  Calculate graph parameters y=ax+b or higher order: b= Hgb (t=0), a=
gradients
  If change in grad than calculate : Grad C-Grad A <= 1.2 gr/dl then = Grad
CORRECTION. Calculate graph with Grad CORRECTION and correct future results.
then continue. If not, activate an alarm.
  If no change in grad then correct results via previous Grad CORRECTION.
  t=Yx20 min (Y=2,3,4 etc.) : Measure and record Hgb levels - If Hgb below X
gr/dL then alarm and continue
  Calculate change in Hgb levels. - If Hgb below _{——} gr/dL then alarm and
continue
  If No change - continue.
    If Positive change - check again and continue. Check for error.
    If Negative change - continue graph. Calculate gradient. If gradient < M
alarm and continue. If not, continue.
```

Detailed Description of Device Components (without Limitations):

The device uses an optical sensor for transmitting radiation known to be absorbed by Hgb. The radiation is detected by a detector, photodiode.

Blood for testing is drawn from the blood flow into a reservoir using a type pump. The data from the device is transmitted to the “display” via Blue Tooth or other communication methods. The device will be power ON by switch embedded in the device.

The frequency in which the blood is being tested is controlled by an automated switch turning the device battery on and off.

The device is stationed stably and tightly on the arm using strips and will allow a flexible assembly on the Venflon.

Optical sensor system: The sensor is based on a photodiode with high sensitivity and a constant working point. The transducer is a LED with more than 1 WATT working in constant voltage. Since the blood sample varies from being dilute to thick liquid, the electrical current of the transducer is increased until the sensor reaches its working point by attenuator current control. This method allows avoiding saturation.

The LED is monochromatic and transmits wavelength at approximately 550 nm which is Hgb isosbestic point. At this point oxyhemoglobin, hemoglobin and carboxyhemoglobin have the same absorption coefficient and allow improved accuracy of the measurements (3% error) with minimal number of wavelengths used to test Hgb concentration. The absorption of light transmitted through the blood sample is correlated to the concentration of Hgb levels in the blood. Using a calibrated plot the Hgb levels can be extracted from the measurements and monitored over time.

The wavelength(s) used depends upon the parameter to be detected. One non-limiting example is that of hemoglobin, wherein hemoglobin is detected at around 550 nm, and wherein the at least one LED outputs radiation at around 550 nm. This wavelength is found to match the Isosbestic point of hemo, oxy and carboxyhemoglobin with identical absorption coefficient (extinction coefficient).

Since we only need the sum of the Hb derivatives, we can radiate the blood at the isobestic point only. Other forms of hemoglobin derivatives are assumed to be less than 3% of the Total hemoglobin concentration in most cases. If better accuracy is needed, more LEDS with additional wavelengths may be used.

Blood Pump:

The blood is drawn from the body using a pump in intervals.

Cuvette:

According to some embodiments, the cuvette is a flow through cell consisting of the blood sample allowing the blood to go through it from the Venflon to the waste. The cuvette is made from a transparent material in the visual range such as quartz, glass, plastics such as PMMA, polystyrene, polycarbonate, or other similar materials. The cuvette may be internally coated with an anti-coagulating coating such as heparin.

The cuvette is manufactured using injection molding or metalworking. The cuvette house is from a non-reflective and rigid material and prevents stray light from escaping or entering the cuvette. Typically, there is an approximately 1 mm diameter hole for light passage.

The optical path (cuvette thickness) ranges from 0.1-2 mm with two parallel faces. The cuvette shape should avoid blood clots and hemolysis. This is done by creating a continuous and constant passage of blood through the cuvette and minimal amount of unintentional chinks.

The cuvette dimensions (height and width) are large enough, so minimal amount of light is scattered in the boundaries later reaching the detector.

Outside this area the cuvette can be either transparent or opaque, as long as it blocks reflected radiation from entering the blood sample again. The cuvette withstands pressure of at least 1.5 bar.

Electrical Stopcock:

Electrical stopcock/4-way electrical valve: The system is mounted to a catheter (venflon) via stopcock (luer). The stopcock is connected to an infusion bag and to the cuvette. The stopcock allows regular body infusion irrigation. When a command from the artificial intelligent system is given (see algorithms), the valve stops the irrigation and allows the pump to draw blood through cuvette from the catheter. After wards, the valve allows flushing the whole device by infusion. After the system is flushed, the sensor checks that the cuvette is clean (signal—base line) and the valve is switched back to regular body irrigation. The infusion may include saline and heparin to lower the risk of blood coagulating in the system and in the catheter. The stopcock is also a check valve preventing the return of drawn blood to the body.
Waste: The blood and infusion fluid is pumped to a waste reservoir.
Data management: Before operation the system calibrates itself and sets a reference point. Raw voltage data can be transferred to a new memory component if needed and continue to work from the last point the device stopped
Performance of the device:
Advantages:
1. Laboratory accuracy of blood indices measurement achieved for whole blood, small blood volume, real-time, and low cost.
2. Immediate results
3. Blood is drawn and tested according to body's behavior using an automatic blood draw algorithm.
4. A reliable and stable access to blood stream, allowing real time blood pumping and monitoring. No blood clots or vessels collapse occur due to the infusion rinsing and dripping procedure.
Methods: Optical spectroscopy, online blood pumping, automatic blood monitoring, hemodilution calibration method

Attained results and analyses: The results and their analysis are attained on-line in less than 1 minute. For low Hgb levels, the alarm will immediately alert the staff. When there are normal Hgb levels but negative Hgb trend, the time it will take for the device to detect the bleeding depends on the rate at which the Hgb levels respond to the bleeding. For example, in acute bleeding situations, Hgb levels will change in less than 20 minutes and the device will alert accordingly. At lower bleeding rates, detection time might be longer.

The references cited herein teach many principles that are applicable to the present invention. Therefore the full contents of these publications are incorporated by reference herein where appropriate for teachings of additional or alternative details, features and/or technical background.

It is to be understood that the invention is not limited in its application to the details set forth in the description contained herein or illustrated in the drawings. The invention is capable of other embodiments and of being practiced and carried out in various ways. Those skilled in the art will readily appreciate that various modifications and changes can be applied to the embodiments of the invention as hereinbefore described without departing from its scope, defined in and by the appended claims.

1-18. (canceled)

19. A system for real-time monitoring and repetitive measuring of whole blood, the system comprising:
a) a fluid delivery device in fluid connection, at a first end, with a blood vessel of a subject;
b) a monitoring and measuring apparatus for direct real-time measurement of at least one parameter in a whole blood sample conveyed by said fluid delivery device from said subject; and
c) a processor adapted to analyze data received from said monitoring and measuring apparatus to detect changes in said at least one parameter of said subject over time.

20. The system according to claim 19, further comprising a valve device at a second end of said fluid delivery device;

21. The system according to claim 19, wherein the processor is further adapted to provide an alarm if bleeding is detected.

22. The system according to claim 19, wherein said whole blood sample is of a volume of less than 150 microliters.

23. The system according to claim 19, wherein the system examines blood by command sent by an external source or an artificial intelligence system and wherein the system is capable of operating continuously for at least 6 hours.

24. The system according to claim 19, wherein the system draws blood via a pump and wherein the pump works in pulses with a time control via a blood sensor and an artificial intelligence system.

25. The system according to claim 19, wherein the system further comprises an optical sensor including at least one LED.

26. The system according to claim 19, wherein hemoglobin is detected between 535 and 560 nm, and wherein at least one LED outputs radiation at around 550 nm.

27. The system according to claim 19, wherein the sensor is a photodiode placed in front a LED and therebetween is disposed a cuvette, a PD setpoint is determined at device initialization, at empty and filled states, wherein a working point is at the middle of a dynamic range of the sensor, at each state; and wherein during sampling, the LED intensity is automatically changed in defined steps until the PD reaches its determined initial setpoint.

28. A diagnostic method for detecting at least one change in a trend of a whole blood parameter, the method comprising monitoring at least one whole blood parameter selected from at least one of: a hemoglobin level, an albumin level, an oxygen level, a sodium level, a potassium level, and pH and combinations thereof of a patient's whole blood; whereby at least one trend is monitored so as to detect one or more changes in said at least one dynamic trend in said whole blood.

29. The diagnostic method according to claim 28, further comprising providing a wearable device for monitoring said change in trend of the whole blood parameter.

30. The diagnostic method according to claim 29, comprising continuously monitoring only a hemoglobin level.

31. A diagnostic method for detecting at least one change in a trend of a whole blood parameter, the method comprising:
a. monitoring and transmitting at least one whole blood parameter; and
b. detecting at least one of a hemoglobin level, a sodium level, an oxygen level, a potassium level, a pH and combinations thereof in a sample of blood;
whereby at least one trend and at least one parameter is monitored from whole blood, without dilution thereof, so as to detect one or more changes in said at least one trend to reflect at least one of internal bleeding, external bleeding and combinations thereof in said patient.

32. The diagnostic method according to claim 28, further comprising providing an alarm if bleeding is detected.
